(19) United States
(12) Patent Application Publication
(10) Pub. No.: US 2021/0338149 A1
(43) Pub. Date: Nov. 4, 2021

(54) MEDICAL DEVICE PROBE FOR AUGMENTING ARTIFICIAL INTELLIGENCE BASED ANATOMICAL RECOGNITION

(71) Applicant: Richard L. Angelo, Seattle, WA (US)

(72) Inventor: Richard L. Angelo, Seattle, WA (US)

(21) Appl. No.: 16/866,133

(22) Filed: May 4, 2020

(51) Int. Cl. A61B 5/00; G06N 20/00; G06N 5/04; A61B 1/317; A61B 5/107; A61B 34/20; A61B 1/00

(52) U.S. Cl. CPC A61B 5/4576; G06N 20/00; G06N 5/04; A61B 1/317; A61B 5/1077; A61B 2090/395; A61B 5/7267; A61B 34/20; A61B 5/6851; A61B 1/00045; A61B 5/7455

(57) ABSTRACT

A surgical probe for augmenting artificial intelligence (AI) based anatomical recognition. Preferably the medical device probe is applied to any body cavity or space for which an image can be obtained and the medical device probe delivered to the target tissue captured on the image (i.e. endoscopic, arthroscopic, or other minimally invasive surgical procedures) in order to train and augment anatomical recognition models. The probe may provide reference points, markings, surface contours, size and scale representations, dye marking, spatial cues, light refraction, sonic propagation and other modalities for determining tissue properties and biomechanical characteristics. A data set of device probe augmented medical images and video are captured from the procedure and used to train the AI based recognition algorithms. The probe, which is designed with a machine-recognizable shaped tip and scale of reference markings, is manipulated by the surgeon to outline or “paint” specific tissue sites, explore tissue makeup, and provide other augmentations to the medical imaging dataset for AI recognition model development. The trained AI model provides immediate feedback with regard to anatomical feature identification, size, biomechanics, surgical guidance and disease state diagnosis.

### BACKGROUND

Medical devices and techniques that enhance minimally invasive surgical procedures and refine diagnoses are available and employ medical imaging, arthroscopy, endoscopy, laparoscopic surgery, and robotic assisted surgical methods. Preoperative medical imaging may be used by the surgeon for diagnosis and assistance with surgical planning. During arthroscopic surgery, the patient's joint, such as a shoulder or knee, does not have to be exposed through a large incision, but is approached through a few tiny incisions or portals that provide access to the involved joint for surgical instruments and the arthroscope. Similarly, during laparoscopic surgery, the abdomen or pelvis is also examined through portals established with small incisions. For the practice of endoscopy, the practitioner examines the inside of the patient's gastrointestinal tract with a camera and fiber optic light source. Small incisions also facilitate the use of Robotic methods to allow the surgeon to operate using the tiny articulated robot arms to produce more precise movements, often in spaces difficult to access.

There are efforts in computer-assisted surgery for planning and guiding surgical procedures. Medical imaging with CAT scans, MRI, x-ray and ultrasound are used to develop computerized models of the patient's body and disease state. The medical imaging dataset may then be used by the surgeon to diagnose, plan and program surgical robots to assist with the components of an operation. Surgical navigation techniques are also used for precise localization of bone structures using medical imaging data, digital cameras, and reference points or light emitting markers.

The current state of the art with minimally invasive surgeries, endoscopic procedures, arthroscopy, and robotic methods, is predominantly limited to pre-operative diagnosis and planning. There are some efforts in using computer assisted technologies in real time during surgical procedures; such as with robotic surgery and CAT scan data to assist with making real-time surgical decisions and to affect the actual extent of bone resection, etc. However, the readily available medical imaging data available during surgery is not being fully exploited. The currently described system is designed to leverage the high value of data that is dynamically gathered during surgery and provide the surgeon with immediate anatomical recognition, feedback, guidance, measurement, and diagnosis of tissue properties and disease states throughout the body.

### SUMMARY

In a preferred embodiment, the system comprises an integration of the recognition and measurement of anatomy through artificial intelligence (AI) modeling with surgeon-directed input to the models using a physical probe to identify and mark specific anatomical sites. The AI modeling is facilitated by the machine learning and recognition of anatomy, pathology and surgical instrumentation, using dynamic medical imaging provided by the endoscope during surgical procedures. The surgeon augments the AI by identifying and marking specific anatomical features, or sites for recognition by the AI models (i.e. bone loss, cartilage defects, tendon gaps, etc.) using a specialized surgical probe. The probe embodies unique features that enhance recognition by the AI models. The probe provides input into the AI system for calibration and measurement using the reference data supplied. Precise anatomical reference points, tissue dimensions, density, biomechanical properties, and pathologies are provided to the system by the surgeon for training the AI model and obtaining real time feedback.

With limitations in machine vision, and displacement or differences in the apparent position of an object in the body, or overlapping tissues, such as cartilage over bone, pure AI approaches may fail to accurately recognize anatomical patient features (i.e. bone loss, defect size, or extent of tissue tearing). By augmenting AI with the manual input from a surgeon and a specialized surgical probe, machine vision techniques more precisely recognize chosen reference points. That data is used to map the location, dimensions, size, shape and condition of different types of tissue. The probe may preferably be embodied with a color, shape, or dimension readily recognizable by the AI models on the medical imaging provided by the endoscopic views obtained during the procedure. The probe may preferably be embodied in a be flexible or rigid form factor, and delivered to any human body space, cavity, lumen, region (hollow or solid) by numerous techniques, including a portal through tissues, endovascularly through a cannula, or through a needle, or other cannulated device, such that it may be introduced into a specified body space, cavity, or tissue (hollow or solid) that can be imaged in any manner recognizable by artificial intelligence (AI), or machine vision. A retractable, sheathed application may be affixed to a physical location of interest, and the probe tip exposed by retraction for recognition by the medical imaging system. Alternatively, the surgeon may apply an inert dye or intra-articular mark to identify specific anatomical reference points in the body or joint using the probe for identification by the A.I. system.

By providing points of reference and spatial cues, the surgeon is able to train the AI model to precisely identify and recognize areas of interest inside the patient's body during surgery. The advantages are the immediate and precise feedback and calculation of tissue properties and the diagnosis of disease states by the system. With probe-derived input from the surgeon, the AI is able to provide accurate real time percentages of tissue loss in specific areas of the body, such as bone defects in the glenoid of the shoulder. Further, the probe may enable the A.I. to map tissue depth and integrity. The system provides a highly valuable method to augment the AI models with data points provided by the surgeon employing the specialized probe in return for the rapid assessment and computation of tissue properties.

### DESCRIPTION OF THE DRAWINGS

FIG. 1 is an embodiment of an endoscopic probe medical surgical device with reference ball tip and calibration markings to provide reference points for determining tissue size and biomechanical properties; the dimpled, spiked ball tip provides texture to increase recognition by the AI powered medical imaging system; preferably, the probe tip may map out margins of tissue, void, etc. (in this instance, a bone defect on the glenoid face and approximate native anterior glenoid rim); the system software calculates approximate area of bone loss; and the shaft of the probe may be calibrated with markings or discs at regular specified intervals.

FIG. 2 is an embodiment of an endoscopic probe with a spiked tip to secure the probe to the target anatomy; the tip is preferably colorized, shaped and textured for recognition by the medical imaging system AI; the ball tip is covered with a retractable sheath, and exposed when the sheath is retracted; a sliding knob on the device handle advances and retracts the probe sheath; the probe tip is recognized by the AI image capture when the operator elects to have a site identified.

FIG. 3 is an embodiment of an endoscopic probe for non-aqueous dye marking; a central piston dispenses a predetermined amount of dye by activating the trigger on a one way valve and dye reservoir; the marker tip is shaped with a finned membrane window that opens with pressurization of the dye chamber by the piston; alternatively, the device may provide a flexible button on the probe marker handle that pressurizes the dye chamber to deliver a defined aliquot of dye from the marker tip.

FIG. 4 is an embodiment of the system for augmenting artificial intelligence (AI) based anatomical recognition; the arthroscope is inserted into the joint (shoulder) through a cannula; the feed from the arthroscope is connected to the camera box and attached monitor; which displays the interior of the shoulder joint; the artificial intelligence models and algorithms identify and label the anatomical features, as augmented and mapped by the medical device probe; and the AI provides calculation of anatomical feature size, dimensions, surface area loss, defect area, and estimated volumetric size and loss.

### DETAILED DESCRIPTION

In a preferred embodiment, the system precisely calculates the size, properties, and characteristics of anatomical features by augmenting artificial intelligence (AI) powered medical image recognition with human input, reference point marking, tissue exploration and training. Unaided medical imaging recognition, does not provide reliable and accurate tissue property assessment, due to limitations with depth perception, parallax, and shortcomings of the technology. By providing human surgeon augmentation of AI-powered anatomical feature recognition, during surgical procedures, the resulting computation of tissue properties is exponentially increased in accuracy and reliability. The surgeon preferably examines and explores the patient's tissues, (i.e. bone and cartilage, soft tissue defect, lumen, etc.) with a probe for marking areas of interest and providing a conduit for feature recognition by medical imaging systems. A preferred example is the surgeon scoping, tracing, and mapping out with reference points and contour following gestures, an area of tissue loss for recognition and computation by the system.

During or subsequent to the capture of endoscopic imaging data, the medical device probe is manipulated by the surgeon to examine and identify specific anatomical regions, surgical sites, or other areas of human anatomy. In a preferred embodiment, the device probe is constructed of a shaped and contrasting color pattern for recognition by the AI powered medical imaging system. The probe may be mechanically obscured with a retractable sheath to hide the probe from view; and whereas the surgeon or operator retracts the sheath to expose the probe for recognition and capture by an arthroscopic camera or imaging device. Preferably, the probe tip and image capture device are positioned and arranged at the specific anatomical feature for optimized augmentation, observance, identification, recognition and diagnosis of tissue properties by the health care provider and associated AI algorithms.

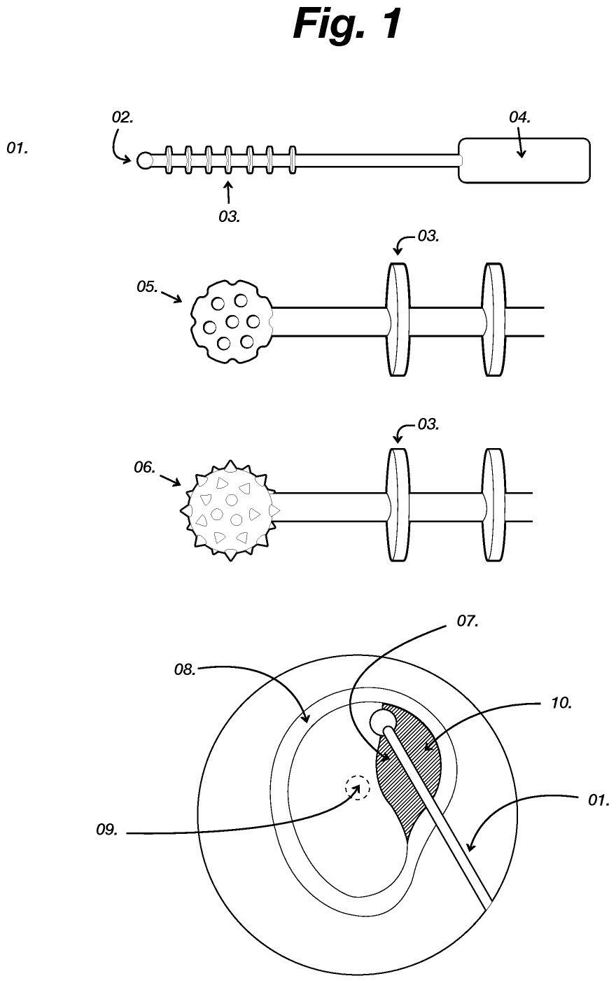

In FIG. 1, a preferred embodiment of the medical device probe or endoscopic probe is shown with a tip of various shapes, such as a sphere, cube, triangle, ring, etc., and additionally, with various textures, such as spiked, dimpled, roughened, etc, and with various contrasting colors or color patterns; all in order to optimize recognition by the artificial intelligence (AI) system and algorithms. The shaft of the medical device probe may preferably be calibrated with markings or discs at regular specified intervals. Additionally, a view of the arthroscopic shoulder glenohumeral joint is shown (as a preferred example), as viewed from an anterosuperior portal with inferior at the top and superior at the bottom of the image; the glenoid is represented with an anterior inferior bony defect and an associated Bankart lesion (anteroinferior capsulolabral tear); the medical device probe is contour mapping or touching the anteroinferior aspect of the glenoid defect to mark its inferior extent; and with additional mapping by the probe, outlines the confines of the defect permitting calculation of the area of bone loss.

In a preferred mode of operation, during or subsequent to the capture of endoscopic images and data set, the medical device probe is employed to identify specific anatomical features, sites, tissue, bone, etc., or regions of human anatomy. The medical device probe is uniquely constructed to provide a size, shape, configuration, texture, color, and pattern in order to optimize recognition by the artificial intelligence (AI) models and algorithm. The probe may be embodied in a flexible or rigid design, and may be delivered to any body space, cavity, lumen, region (hollow or solid), by portal through tissues, endovascularly through a cannula, or through a needle or cannulated device, such that it may be introduced into a specified body space, cavity, or tissue that can be imaged in any manner recognizable by artificial intelligence (AI), or machine vision. Preferably, a sheathed medical device probe, obscures the probe tip from view. When a specific anatomical feature or site is to be identified, the operator retracts the sheath to expose the probe tip for capture by the endoscopic imaging device and recognition by the system AI. The operator preferably manipulates the probe to outline or “paint” specific regions of the anatomy; and with the captured medical imaging data, and other sensor data, immediate calculations of length, width, area, and volume may be obtained. Additionally, the probe may be utilized to assist the AI model in identifying regions or sites of anatomy that may lack distinguishing features and are otherwise more difficult for the models to consistently identify. The medical device probe may also preferably include a probe tip, which dispenses an aliquot of non-aqueous dye to mark and provide reference points regarding specific anatomical features, or sites for enhanced dye-marked recognition by the system AI.

In a preferred embodiment, the device probe is manipulated by the surgeon to outline or “paint” specific anatomical features or regions. In this respect, the probe is maneuvered or gestured to contour map a region of interest. During an arthroscopic surgery, the surgeon may desire to obtain a precise measurement of a tissue defect or surface area. With the anatomical feature positioned within the imaging system field of view, the surgeon simply exposes the medical device probe tip for recognition and capture by the imaging device, and traces, draws or paints the circumference of the area of bone loss or articular cartilage for measurement. The system AI recognizes and captures the probe tip location, movement pattern, and associated augmentation activity; and in return, provides immediate calculation of the anatomical features of target tissues throughout the body in which a image can be obtained (i.e. endoscopic, ultrasound, or other energy-derived image). Additional properties of tissue length, width, and volume may also be similarly gesture mapped and calculated by the system.

In a preferred embodiment, the surgeon performs arthroscopic reconstruction of glenoid bone loss in the shoulder. Medical imaging with X-ray, CAT-scan, or MRI are typically used to attempt an diagnosis of the size and amount of glenoid bone loss. The estimation and quantification of the amount of bone loss is crucial for operative decision making and patient clinical outcome. With the presently described system, the surgeon provides reference points for measuring the area of bone loss. The surgeon preferably inserts the probe into the joint, via an arthroscopic portal, traces the contours of the glenoid bone for capture by the medical imaging system, as provided with at least one arthroscope. At points of reference along the glenoid bone, the surgeon places the probe along the circumference of the glenoid, provides a marking event, either by clicking a snapshot button, or digitally or physically registering the edge point, and the system captures the data. From the surgeon provided augmentation and input of multiple points along the glenoid bone, the artificial intelligence based medical imaging system computes the precise area and size of glenoid bone loss.

In a preferred embodiment, the surgeon provides reference points with a sheathed medical device probe that is anchored with a sharp tip to fix the device to tissue areas of interest. The surgeon places the probe at an edgepoint along the glenoid bone area, which resembles a circle, and retracts the sheath to expose the device tip marker with a colorized, contrasting, or shaped tip for recognition by the imaging system. Additionally, the surgeon may provide haptic feedback to the probe by clicking a button or actuator to signal to the imaging system that a point of reference event has occurred. The sheath is closed upon completion of the marking event. In this procedure, the AI powered image recognition is exposed to the device tip only during marking events for determining anatomical points of reference.

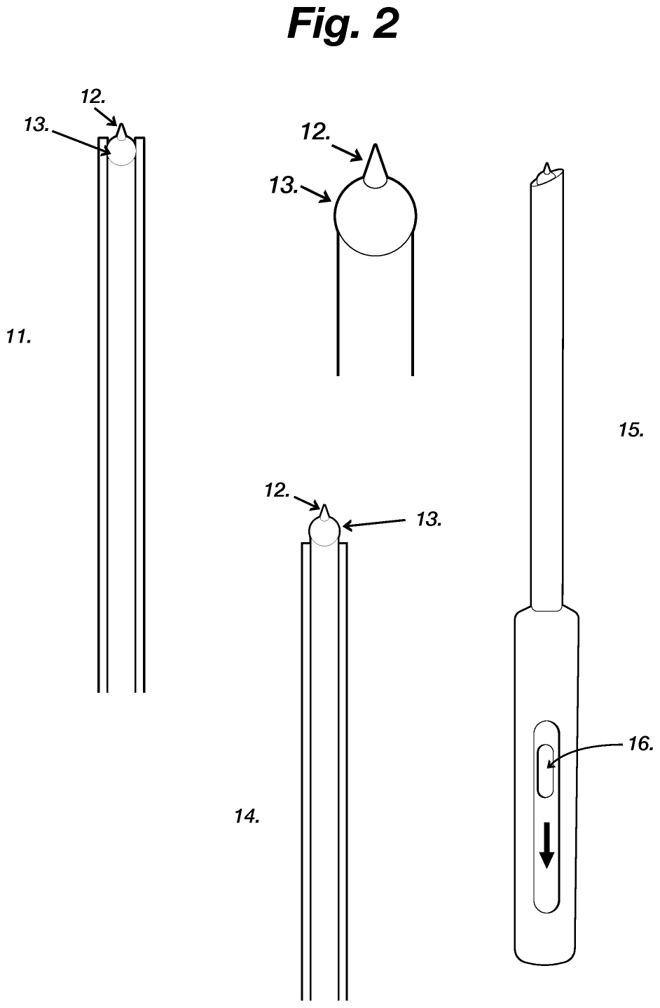

In FIG. 2, a preferred embodiment of the spherical medical device probe is shown with spiked tip, to assist with secure approximation with target anatomy, and a retractable sheath. Preferably, the sheath covers the spherical tip from being visible, and when retracted, permits viewing of the sphere and recognition by the system AI model. The medical device probe handle is shown with sliding knob that advances and retracts the probe sheath. Therefore, the probe is only recognized by the system AI when the operator specifically elects to have a site identified.

In a preferred embodiment, the system is provided with an array of marking events and locations along the glenoid bone area. The data set may preferably be comprised of a set of medical images with device tip exposures along the circumference of the glenoid bone area. The system AI software performs computation of the size and area of bone loss form the data set. From the images and reference points, the glenoid bone circumferential boundary is precisely drawn and surface area is calculated. The distance across the glenoid is preferably calculated by the comparison of at least two images and reference points. The size of the glenoid bone is preferably determined by surface area formula and calibration with the device probe known tip size and other probe reference size markings and visual cues. In a preferred embodiment, the probe tip size is a known three millimeters (3 mm) reference size; and the probe tip may be provided with millimeter ruler markings. Other known probe tip sizes and scale of reference marking may be available depending on the anatomical feature application.

In a preferred embodiment, the system is provided with a compilation of device probe augmented images along the shape, circumference and surface contours of articular cartilage in the knee. The data set may be comprised of a set of arthroscopic image captures showing inert dye markings along the articular cartilage. During the arthroscopic knee surgery, the surgeon provides touch points and applies inert dye to the circumference of the cartilage tissue. The medical imaging dataset, comprised of a set of images with a dye map of the articular cartilage, is computed by the system AI for determining the size, surface area, biomechanical properties, and extent of specified tissue. In a preferred embodiment, with an intra articular marker, the surgeon applies an oil based (or other liquid medium), inert dye to reference points around the articular cartilage. The system AI is provided an augmented dye-mapped image data set for the precise computation of the size of articular cartilage defects.

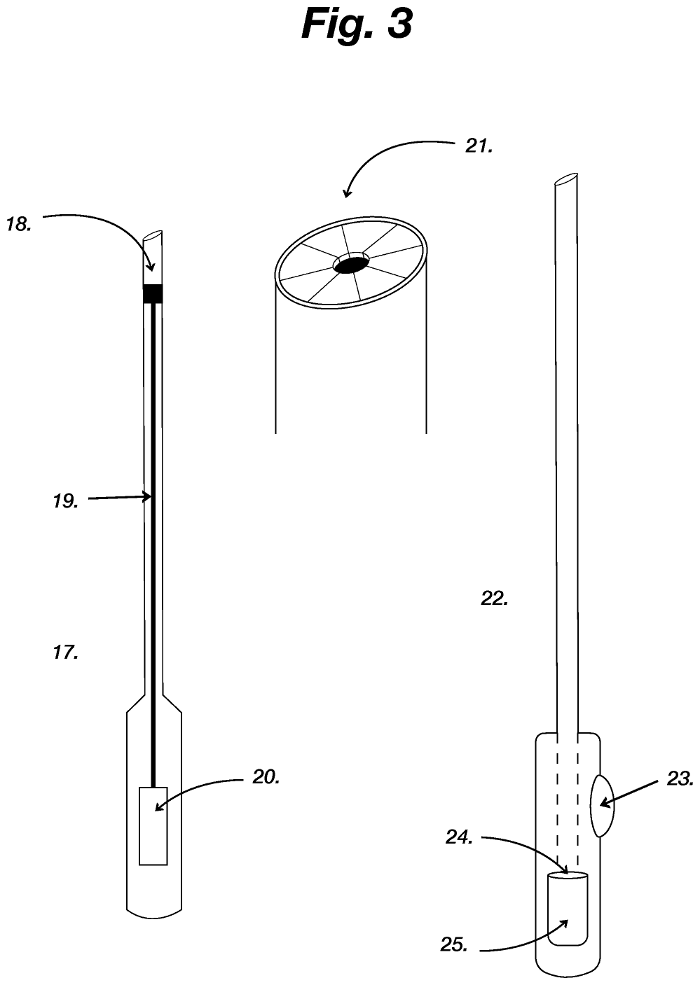

In FIG. 3, a preferred embodiment of the medical device probe endoscopic tissue marker is shown with non-aqueous dye. A central piston is activated using the trigger in a method to dispense one (1) aliquot of dye from the probe or marker tip with each depression of the handle trigger. Preferably, the marker tip may be shaped so as to minimize potential leakage of the dye by virtue of a finned membrane window that opens with pressurization of the dye chamber by the piston. Alternatively, the medical device probe may provide a flexible button on the probe/marker handle, which acts to pressurize the dye chamber, and deliver a defined aliquot of dye from the marker tip.

In a preferred embodiment, the artificial intelligence (AI) model is trained with reference data for the development of recognition algorithms. In this manner, the surgeon performs orthopedic shoulder surgery with medical imaging data being captured by the system from digital camera arthroscopes. The live imaging video feed is delivered to the camera box and displayed on flat panel screens for observation by the surgeon. The medical device probe is inserted into the joint via a portal and the tissues are examined by the surgeon from imaging data available on the screens. Upon setting up a preferred training image of a particular anatomical feature, by adjusting the arthroscopic camera, the surgeon captures the image with the system. The particular anatomical feature may be appropriately labeled by the AI, which has been trained to recognize that feature. Additionally, the medical device probe may be placed at important reference points in the joint for capturing size, shape and other biomechanical properties of the tissue. For example, the probe may have a known tip size of one millimeter (1 mm) and unique shaped feature for recognition, reference, and calibration by the system AI. The surgeon may capture multiple images of anatomical features with the reference probe tip in order to build a reference medical image data set, and train the AI recognition algorithm.

In preferred embodiment, the system AI recognition algorithms are developed and trained with medical imaging data and device probe reference points and calibration levels. As applied to the task of measuring and quantifying glenoid bone loss, the system captures a data set of glenoid bone images with the device probe providing reference location markers and relative size data. The medical device probe is equipped with a colorized, texturized, or shaped tip for AI recognition within the medical images. Preferably, the surgeon places the device probe along the glenoid circumferential areas and captures, or builds a dataset of probe-augmented medical images. In a preferred embodiment, during image capture, the surgeon “draws” or follows the contours of the glenoid circumference, which resembles an elliptical shape. The device tip preferably has a known size for reference by the system AI. The resulting augmented image set provides the AI recognition with reference points describing the elliptical shape of the glenoid, and allows the system to calculate the size of the glenoid and determines the percentage of bone loss. The collection of augmented images provides the AI with a dataset for development of recognition algorithms.

In a preferred embodiment, the system AI recognition algorithms are developed and trained with a data set of medical images depicting various cartilage defects and sizes from arthroscopic knee surgeries, as augmented by the medical device probe. During the procedure, the surgeon looks inside the knee joint with the arthroscopic camera inserted through a small incision or portal. Fluid is pumped into the joint to expand the space between tissues. Additional portals may be used for the insertion of surgical tools and the medical device probe for exploration and examination of the knee joint. The medical device probe may preferably have visible units of length for reference by the system arthroscopic medical imaging capture and AI recognition. For example, the surgeon may place the medical device probe, with visible millimeter markings, along the length of the cartilage defect and capture the image with the arthroscopic camera. Multiple images of the reference probe, as augmenting the image of the cartilage defect, may be captured by the system for building a dataset. The cartilage defect image set, with known size measurements as augmented by the probe, is used by the system AI to develop a cartilage defect size recognition algorithm.

In preferred embodiment, the system AI recognition model, and imaging data capture, is augmented with medical device probe interaction and exploration of the tissue, bone or joint. Tissue depth and integrity may be examined within the joint during the arthroscopic procedure; and it is well know that healthy articular cartilage matrix is much more dense than degenerative cartilage. The medical device probe may assess and capture density measurements with light refraction. Preferably, the probe tip is equipped with a light-emitting (or other energy-emitting) tip and captures the augmentation activity as observed in refraction angle and wavelength measurements of the light as it passes from a medium of known density through the articular cartilage. The medical device may provide a controlled light or other energy source of known intensity, wavelength, or duration, along with a probe tip with known density, refraction, or receptivity properties. An array of imaging device sensor devices may be inserted into the joint for the capture and building of an articular cartilage light refraction data set. The system AI model preferably develops an articular cartilage density measurement algorithm from the data set. The light or energy emitting probe may be applied to any body cavity, space, or lumen of which an endoscopic image may be obtained.

In a preferred embodiment of the system, the artificial intelligence (AI) is provided a training of complex tissue properties with respect to articular cartilage overhang concealing a recessed bone area. With traditional medical imaging or unaugmented AI, machine vision models may struggle with determining if bone is recessed behind cartilage overhang. However, with the presently described system, the AI model is augmented with the surgeon's input as captured by the endoscopic imaging system and tissue manipulation with the medical device probe. In a preferred application, the surgeon provides a training image and video data set showing properties of articular overhang concealing a recessed bone area. The training data set may preferably be comprised of multiple, tens, hundreds or thousands of like images of videos. The medical imaging data may preferably be comprised of exploration of the articular cartilage size, shape, texture, density and other biomechanical properties with the medical device probe, as captured by endoscopic imaging systems. Additionally, the data set may show the structure, layout and composition of articular cartilage overhang, concealing recessed bone. Preferably the surgeon manipulates the medical device probe to lift the cartilage and expose the recessed bone area. The AI model develops algorithms for recognition of articular cartilage overhang and recessed bone area from the medical imaging data set and device probe manipulation and augmentation activity.

In a preferred embodiment, anatomical feature recognition is developed by the system AI with a data set of medical images, videos and device probe reference points, spatial cues, gesture maps, contour traces, and other tissue augmentation activities. Preferably, the AI recognition model is provided a large sample or reference data set of a few hundred, thousand, or more, of device probe augmented medical images for feature extraction and identifying relationships across common anatomical features. For a particular anatomical region of interest, like the shoulder, the images of that region will be classified and labeled according to the specific tissues, bones, ligaments and other features. The data set of sample and reference images should be imported into the AI recognition model for training. The AI recognition model should be provided with a number of objects for recognition, as classified at the specific anatomical region, i.e., the number of different types of bones, ligaments, muscles, cartilages, etc. Additionally, the model may be configured to analyze and train itself on all of the images in the dataset a certain amount of times. Preferably the steps above may be repeated, enhanced, and reconfigured to develop and drive anatomical recognition models of acceptable accuracy and reliability.

In a preferred embodiment, an augmented medical image data set is compiled from arthroscopic shoulder surgery. The data set may preferably be classified and labeled into anatomical categories representing the glenoid, clavicle, scapula, humerus, articular cartilage, synovial membrane, and joint cavity. Each category is preferably comprised of hundreds, thousands, or more, images of the medical device probe augmenting the particular anatomical feature. For example, the images comprising the glenoid cavity data set, may preferably be composed of hundreds of medical images showing the glenoid as referenced by the medical device probe for anatomical feature recognition, size, shape, surface area, and other biomechanical properties. Additionally, the other shoulder joint anatomical features, bones, tissue, and cartilage, may be comprised of a similar augmented medical image data set. The shoulder joint medical imaging data is preferably imported into the AI recognition model for training. The model is preferably configured to perform feature extraction and pattern recognition on all of the images in the dataset; and the process may be repeated, enhanced, and reconfigured to develop and drive acceptable anatomical recognition model.

In a preferred embodiment use case, the augmented medical imaging data set is compiled from arthroscopic shoulder surgery for rotator cuff tears. The data set may preferably be classified and labeled into anatomical categories representing the supraspinatus, infraspinatus, teres minor, acromioclavicular joint, greater tuberosity, coracoid process, and subscapularis. Each anatomical feature category is preferably comprised of hundreds, thousands, or more images of the medical device probe exploring, marking, and providing points of reference and feature identification of the shoulder and rotator cuff musculoskeletal anatomy. In a preferred example, the images comprising the rotator cuff muscles (i.e., supraspinatus, infraspinatus, teres minor), may be comprised of hundreds of medical images showing the specific muscle, and any observable tears or injuries, as examined by the medical device probe. Within each image, the device probe may provide augmented anatomical feature identification, reference markings to tears or injuries, and the characterization of other biomechanical properties. Additional shoulder and rotator cuff anatomical features are likewise comprised of hundreds or more images, identifying the particular feature, with medical device probe augmentation activity. The arthroscopic shoulder and rotator cuff image dataset is preferably imported into the system AI recognition model for training. The model is configured to perform feature extraction, develop pattern recognition; and the process may be repeated, iterated, and reconfigured to develop and drive an acceptably accurate anatomical recognition model.

In a preferred embodiment, an augmented medical image data set is compiled from arthroscopic hip surgery data. The data set may preferably be classified and labeled into anatomical categories representing the femoral head, acetabulum, acetabular labrum, and ligament of the head of femur. Each anatomical category is preferably comprise of hundreds, thousands, or more, images of the medical device probe augmenting and providing reference markings, surface contour, or identification of the anatomy. For example, for the images comprising the femoral head data set, may preferably be comprised of hundreds of medical images showing the highest part of the femur bone as referenced by the medical device probe and captured with arthroscopic imaging devices. Within each image, the device probe may preferably provide reference size, shape, contour mapping, or other biomechanical properties. Each and every other hip joint anatomical feature is likewise comprised of hundreds or more images of the particular feature, with biomechanical properties referenced and augmented by the device probe. The hip joint medical imaging data is preferably imported into the AI recognition model for training. The model is preferably configured to perform feature extraction and pattern recognition on the dataset; and the process may be repeated, enhanced, and reconfigured to develop and drive an acceptable anatomical recognition model.

In a preferred embodiment, an augmented medical image data set is compiled from arthroscopic knee surgery data. The data set may preferably be classified and labeled into anatomical categories representing the tibia, femur, patella, meniscus, ligaments, articular cartilage, quadriceps, and hamstrings. Each anatomical category is preferably comprised of hundreds, thousands, or more, images of the medical device probe exploring, marking and providing points of reference and scale for size of the particular part of the knee anatomy. In a preferred example, the images comprising the meniscus data set, may preferably be comprised of hundreds, of medical images showing a healthy C-shaped, tough, rubbery articular cartilage between the thigh bone and the tibia, as referenced by the medical device probe and imaged by arthroscopic cameras. Within each meniscus image, the device probe may provide augmentation activity showing size and shape of healthy tissue, size of any tears, cartilage density, identification of scar tissue, or other biomechanical properties. The other knee joint anatomical features are likewise comprised of hundreds or more images, identifying the particular feature, with augmentation activity for biomechanical properties. The knee joint data is preferably imported into the AI recognition model for training. The model is configured to perform feature extraction on the data, develop pattern recognition; and the process may be repeated and iterated, and reconfigured to develop and drive an acceptably accurate anatomical recognition model.

In a preferred embodiment use case, the augmented medical imaging data set is compiled from laparoscopic cholecystectomy surgery data. The data set may preferably be classified and labeled into anatomical categories representing the gallbladder, liver, pancreas, stomach, and bile duct. Each anatomical feature category is preferably comprise of hundreds, thousands, or more, images of the medical device probe exploring, marking and providing points of reference and feature identification of the digestive anatomy. In a preferred example, the images comprising the gallbladder image image data set, may be comprised of hundreds of medical images showing a healthy small hollow organ where bile is stored, and any instances of observable inflammation, infection, or the detection of gallstones, as referenced by the medical device probe and imaged by the laparoscopic imaging device. Within each gallbladder image, the device probe may provide augmented references to gallstones location, and areas of inflammation. Other related anatomical features, of the liver, pancreas, stomach, etc., are likewise comprised of hundreds or more images, identifying the particular anatomical feature, with medical device probe identification reference points, dye markings, or other augmentation activity for biomechanical tissue properties. The laparoscopic cholecystectomy image dataset is preferably imported into the system AI recognition model for training. The model is configured to perform feature extraction, develop pattern recognition; and the process may be repeated, iterated, and reconfigured to develop and drive an acceptably accurate anatomical recognition model.

In a preferred embodiment use case application, the augmented medical imaging data set is compiled from endoscopic ultrasound procedures. The data set may preferably be classified and labeled into anatomical categories representing the digestive tract, esophagus, stomach, duodenum, gastrointestinal wall, lymph nodes, and bronchi. Each anatomical feature category is preferably comprised of hundreds, thousands, or more, images of medical device probe exploring, marking and providing points of reference and feature identification of the upper digestive tract or respiratory tract. In a preferred example, the images comprising the pancreas ultrasound from the esophagus, may be comprised of hundreds of medical ultrasound images, as augmented by the medical device probe. Other related anatomical features of the upper digestive tract, as imaged by endoscopic ultrasound, are likewise comprised of hundred or more images, identifying particular anatomical features, with reference points, dye markings, or other augmentation activity as provided by the device probe. The augmented endoscopic ultrasound image dataset is preferably imported into the system AI recognition model for training; and the process may be iterated, and reconfigured to drive an acceptably accurate anatomical reference model.

The system and procedures described here for the medical device probe and AI augmentation may be applied to any human physiological body space or cavity in which a medical image (i.e., arthroscopic, endoscopic, ultrasound, etc.) may be obtained and which the augmentation probe can be delivered to the target tissue. Not only may the surgical probe and artificial intelligence based anatomical recognition system be applied to bone, cartilage and the overall musculoskeletal system; but also to specific organs. In a preferred embodiment, the system may be applied to the heart where an ultrasound image could be obtained and a probe delivered to the heart endovascularly to augment the medical image data to measure the appropriate size for mitral valve replacement. The system may be applied to any body space that may be imaged and probed in order to provide anatomical feature identification, sizing, and measurement of biomechanical properties.

In a preferred embodiment, the medical device probe, in addition to being rigid, may be flexible and similar to a wire introduced through a catheter endovascularly. A bulb at the tip of the wire probe may have a known size, such as two or three millimeters (2-3 mm). When the bulb tip is imaged with an endoscopic or ultrasound image, or other imaging sensor, it may be used as a magnification reference in order to estimate specified distances on the image. The wire probe may be delivered endovascularly into a region of the heart, such as the mitral valve area. Using the two millimeter (2 mm) ball tip of the wire probe as a reference, an accurate estimate may be made using the artificial intelligence (AI) assessed images as to the appropriate size of mitral valve replacement needed. The properly-fitted mitral valve replacement could then be delivered into position and secured.

In a preferred embodiment of the system AI recognition model, the dataset of anatomical features may be comprised of hundreds, thousands, or more, of medical images as augmented by the device probe. Within each image, the surgeon has preferably provided device probe augmentation activity, such as tissue, bone, musculoskeletal, and other anatomical reference points, tissue identification, size scaling, tissue density, and other biomechanical properties. The imaging dataset is preferably developed with metadata for the specific device probe augmentation activity. Each augmented medical image may have associated metadata for anatomical feature, location, patient health data, and any disease state. The recognition model preferably performs pattern recognition, feature selection, detection, extraction, and dimensionality reduction on the the imported dataset. The model may preferably develop and build a convolutional neural network classifier from the dataset. The dataset is preferably further used to train the system AI recognition model; and the process may be iterated, and reconfigured to drive an acceptably accurate anatomical reference model.

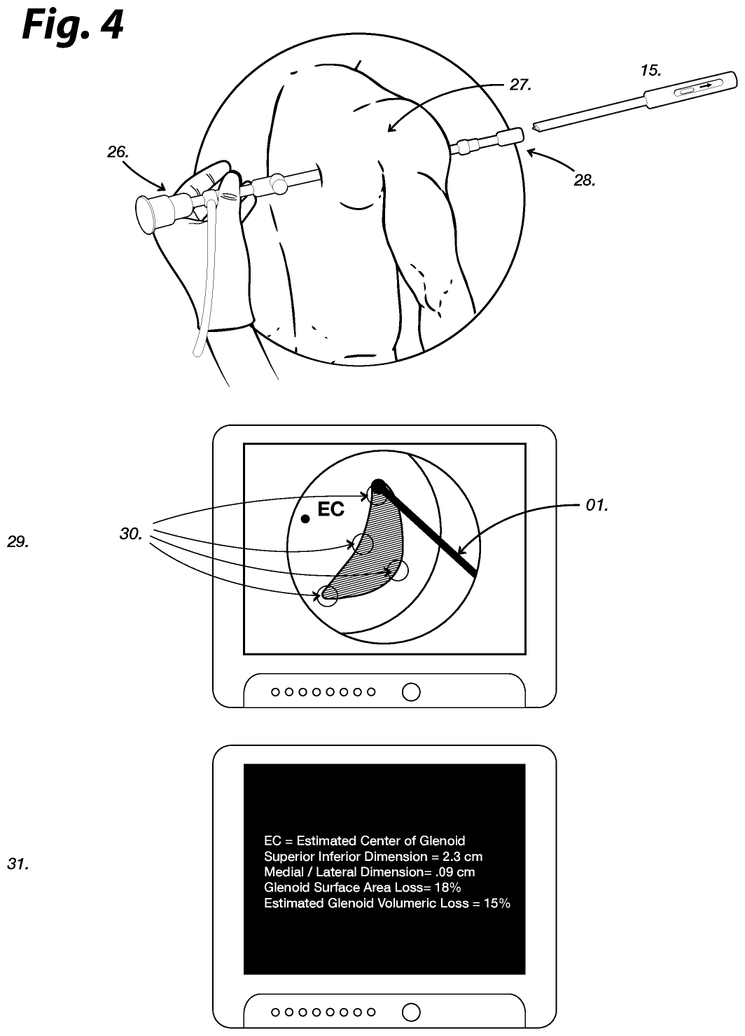

In FIG. 4, a preferred embodiment of the medical device probe for augmenting artificial intelligence (AI) based anatomical recognition, is shown. The arthroscope is preferably inserted into the shoulder through cannulas. The imaging device data is captured by the arthroscopic camera and interfaced with the arthroscopic monitor, which displays the arthroscopic image of the interior of the shoulder joint. The artificial intelligence (AI) model and algorithms identify and label 1) the glenoid; 2) the estimated center (EC) of the glenoid; 3) the Bankart lesion; and 4) the glenoid labrum. The AI system is augmented by the medical device probe, which provides reference points on the Bankart lesion, including the superior, inferior, medial, and lateral extent of the glenoid defect. The medical device probe in FIG. 4 identifies the inferior extent of the Bankart lesion. The AI model and algorithm, additionally measures the distance between the points identified by the probe; where the superior/inferior distance is calculated as 1.2+1.1=2.3 cm; and the mediolateral distance is at 0.9 cm. Additionally, on another display device or monitor, the AI system, model and algorithm, notes the EC (or estimated center of the glenoid based on cumulative arthroscopic views and image data sets) and calculates the Superior/Inferior Dimension at 2.3 cm; the Mediolateral Dimension at 0.0 cm; the Glenoid Surface Area loss at eighteen percent (18%); the Glenoid Defect Area at 2.07 cm-sq; and the Estimated Glenoid Volumetric Loss at fifteen percent (15%).

In a preferred embodiment of the medical device probe, the probe may be constructed out of autoclavable surgical grade materials, 316 stainless steel, titanium, nickel-clad construction, plastics, ceramics, polymers, or other biomaterials appropriate for the specific medical procedure. Alternatively, the medical device probe may be constructed out of biocompatible materials, and be of a single use, disposable design. The design of the probe tip may be of a shaped sphere, angular, hexagonal, triangular, or other defined contours for recognition and reference by the system AI. The retractable sheath configuration may be preferably be constructed in either a metallic or plastic form factor. The device handle is configured, depending on surgical application, to fit an optimized ergonomic position for the surgeon's hands, and may be embodied in a slightly curved handle, t-handle, straight handle, or other design. The retractable sheath, haptic feedback button, dye dispensing button, event marker, and other augmentation activity feedback may be integrated into the device handle for pairing device probe manipulation and feature mapping with medical imaging sensor data.

1. A medical device probe system and method for calculating the size of glenoid bone loss comprising the steps of:
streaming live arthroscopic medical images during shoulder surgery;
inserting a medical device probe for evaluation of glenoid pathology;
tracing contours of glenoid bone;
capturing a plurality of marking events and reference points of the glenoid pathology with a medical imaging system; and
computing a size and area of glenoid bone loss;
wherein, an artificial intelligence based anatomical recognition system is augmented and calibrated with a known medical device probe tip size and reference markings; wherein, the system captures images of a glenoid bone circumference; wherein, a distance across the glenoid is calculated by the comparison of marking events, reference points, images and probe size; and wherein, the size and area of glenoid bone loss is computed from surface area calculation.

2. The medical device probe system and method for calculating the size of glenoid bone loss of claim 1, further comprising the steps of:
anchoring a sheathed medical device probe at an edgepoint along a glenoid bone area; and
retracting a sheath to expose a device tip for recognition by the imaging system.

3. The medical device probe system and method for calculating the size of glenoid bone loss of claim 1, further comprising the steps of:
providing haptic feedback to the medical device probe for signaling to the imaging system that a marking event or reference point has occurred.

4. The medical device probe system and method for calculating the size of glenoid bone loss of claim 1, wherein the plurality of marking events and reference points of the glenoid pathology comprise a set of medical images with device tip exposures along a circumference of the glenoid bone area.

5. The medical device probe system and method for calculating the size of glenoid bone loss of claim 1, further comprising the steps of:
applying inert dye markings to reference points around the glenoid pathology; and
providing the artificial intelligence based anatomical recognition system with an augmented dye-mapped image data set.

6. The medical device probe system and method for calculating the size of glenoid bone loss of claim 1, wherein the artificial intelligence based anatomical recognition system is trained with medical imaging data comprising labeled anatomical features and known device tip size.

7. The medical device probe system and method for calculating the size of glenoid bone loss of claim 1, further comprising the steps of:
developing an anatomical recognition algorithm with a data set of medical images as augmented by the medical device probe;
wherein, a surgeon builds the data set by capturing images of the device probe along glenoid circumferential areas.

8. A medical device probe system and method for anatomical recognition comprising the steps of:
streaming live medical images during minimally invasive surgery;
applying a medical device probe to an anatomical feature;
exploring biomechanical properties of the feature with the probe;
capturing a plurality of marking events and reference points of the feature with a medical imaging system; and
generating anatomical feature identification and recognition;
wherein, an artificial intelligence based anatomical recognition system is augmented and calibrated with a known medical device probe tip size and reference markings; wherein, the system captures images of the anatomical feature; wherein, the size of the feature is calculated by the comparison of marking events, reference points, images and probe size; and wherein, the identification and recognition of the anatomical feature is computed from captured medical images and known reference images.

9. The medical device probe system and method for anatomical recognition of claim 8, further comprising the steps of:
anchoring a sheathed medical device probe at an edgepoint along the anatomical feature; and
retracting a sheath to expose a device tip for recognition by the imaging system.

10. The medical device probe system and method for anatomical recognition of claim 8, further comprising the steps of:
providing haptic feedback to the medical device probe for signaling to the imaging system that a marking event or reference point has occurred.

11. The medical device probe system and method for anatomical recognition of claim 8, wherein the plurality of marking events and reference points of the anatomical feature comprise a set of medical images with device tip exposures along a circumference of the feature.

12. The medical device probe system and method for anatomical recognition of claim 8, further comprising the steps of:
applying inert dye markings to reference points around the anatomical feature; and
providing the artificial intelligence based anatomical recognition system with an augmented dye-mapped image data set.

13. The medical device probe system and method for anatomical recognition of claim 8, wherein the artificial intelligence based anatomical recognition system is trained with medical imaging data comprising labeled anatomical features and known device tip size.

14. The medical device probe system and method for anatomical recognition of claim 8, further comprising the steps of:
developing an anatomical recognition algorithm with a data set of medical images as augmented by the medical device probe;
wherein, a surgeon builds the data set by capturing images of the device probe along the anatomical feature.

15. A medical device probe system for anatomical recognition comprising:
a medical imaging capture device;
a surgical probe for augmenting medical imaging data;
an algorithm for comparing probe augmented imaging data with a labeled data set of anatomical features; and
an artificial intelligence model for providing anatomical feature identification and biomechanical properties;
wherein, the imaging data is augmented by surgical probe marking events and reference points; and wherein the artificial intelligence model is trained with medical imaging data comprising labeled anatomical features and known probe characteristics.

16. The medical device probe system for anatomical recognition of claim 15, wherein the marking events and reference points comprise a set of medical images and probe evaluations of the anatomical feature.

17. The medical device probe system for anatomical recognition of claim 15, wherein the artificial intelligence model is developed with a data set of medical images as augmented by the medical device probe.

18. The medical device probe system for anatomical recognition of claim 15, wherein the labeled data set is built by capturing images of the device probe along known anatomical features.

19. The medical device probe system for anatomical recognition of claim 15, wherein anatomical feature identification is provided in real time with the capture of probe augmented medical imaging data.

20. The medical device probe system for anatomical recognition of claim 15, wherein biomechanical properties are provided in real time with the capture of probe augmented medical imaging data.
